(19) United States
(12) Patent Application Publication
(10) Pub. No.: US 2022/0062141 A1
(43) Pub. Date: Mar. 3, 2022

(54) PERSONAL CARE COMPOSITIONS AND USES THEREOF

(71) Applicant: Zhengmei HUANG, Wuxi (CN)

(72) Inventor: Zhengmei HUANG, Wuxi (CN)

(21) Appl. No.: 17/060,000

(22) PCT Filed: Mar. 26, 2019
(86) PCT No.: PCT/CN2019/079697

§ 371 Date: Nov. 30, 2020

(51) Int. Cl. A61K 8/60; A61Q 1/02; A61Q 19/10; A61K 8/73; A61Q 19/00; A61K 8/92; A61K 8/9794; A61K 8/99; A61K 8/34; A61K 8/44

(52) U.S. Cl. CPC A61K 8/60; A61K 2800/33; A61Q 19/10; A61K 8/732; A61Q 19/007; A61K 8/922; A61Q 19/00; A61K 8/9794; A61K 8/735; A61K 8/99; A61K 8/345; A61K 8/44; A61K 2800/524; A61K 2800/30; A61Q 1/02

(57) ABSTRACT

The personal care compositions or cosmetic systems using reduced level or no preservative and/or surfactant, including skin care essences, lotions, creams and moisturizing liquids and uses thereof.

### TECHNICAL FIELD

The present disclosure relates to personal care compositions with reduced water activity and uses thereof. In particular aspects, provided herein are personal care compositions or cosmetic systems using reduced level or no preservative and/or surfactant, including skin care essences, lotions, creams, moisturizing liquids, cleansers and uses thereof.

### BACKGROUND

In the following discussion, certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Personal care products are susceptible to microbial contamination during production, storage, and use, which in turn leads to deterioration of the product and produces some irritating and harmful ingredients that affect human health. Therefore, how to effectively reduce or prevent products from microbial contamination is particularly important for the safety of personal care products.

In general, appropriate amounts of preservatives are added to personal care products to prevent the growth of microorganisms. Antiseptic and bacteriostatic agents commonly used in cosmetics can be classified into four categories according to their chemical structures: alcohols, formaldehyde donors and aldehyde derivatives, benzoic acid and its derivatives, and other organic compounds. The main products are phenoxyethanol and benzyl alcohol, imidazolidinyl urea, parabens, bromopol, carson and quaternary ammonium salts, and the like. These preservatives inhibit the cell division and inhibit the growth and reproduction of the cells by acting on multiple targets such as cell membranes, cell walls and enzymes of microorganisms, thereby achieving the purpose of antisepsis and bacteriostasis. (Zheng Xingquan, Zhou Shu, Zhou Wei. Cosmetics Hygiene Inspection Manual [M]. Beijing. Chemical Industry Press, 2003: 369-370)

However, the use of preservative-added cosmetics may cause various adverse reactions to the skin after use, and long-term use may also disturb the resident flora of the skin, thereby changing the micro-ecological environment of the skin. Changes in pH, humidity, oil content, etc., affect the secretion function of sweat glands and sebaceous glands, and hinder the self-purification and metabolic functions of the skin. (Chen Guanwu. Study on the inhibitory effect of Jiema BP on the surface layer of resident bacteria[J]. Journal of Environment and Health, 2012,29(7):615-616).

Jima BP is a mixture of diimidazolidinyl urea (also known as Jema) and IPBC. Studies have shown that its aqueous solution is added to cosmetics to inhibit P. acnes and Staphylococcus epidermidis, and it has a certain effect on the resident bacteria of the skin. The short-term microbial challenge test was used to determine the inhibitory effect of aqueous solution of methylparaben and methylparaben-containing cosmetics on Propionibacterium acnes and Staphylococcus epidermidis. The results also showed that methylparaben added to cosmetics. The resident bacteria of the skin produce a certain inhibitory effect. (Cai Ying. Inhibition of skin resident bacteria by methylparaben[J]. Modern Preventive Medicine, 2013,40(9):1726-1727).

Cosmetics are often prone to microbial contamination, mainly in cosmetics containing high moisture and various nutrients suitable for microbial growth, or in the production, canning, easy to bring in water or microorganisms caused by microbial contamination during use. Deteriorated products cause form, odor, and color changes, which in turn cause skin irritation and irritation to consumers. The high-moisture cosmetic system is suitable for consumers use, but more anti-corrosion agent systems are needed for using at different temperatures and under different use environments. The frequent opening of the anti-corrosion system to air also requires more stringent anti-corrosion capability for the cosmetic products.

At present, skin care products (e.g., essences, lotions, creams) in the personal care industry use preservatives to address the potential microbial contamination. Many types of preservatives, whether listed in the INCI list or not, have been used in current skin care products. Since skin care products are applied to the skin, the skin care products need higher water content. The higher water content and nutrients in the skin care ingredients often cause microbial growth in the skin care products. Therefore, the current skin care products predominately use preservatives to inhibit the growth of microorganisms. The preservatives used in current skin care products, however, harm and in some cases, destroy the microbial ecosystem of user's skin, and cause growing number of skin problems.

There is a growing need for personal care compositions, including skin care compositions and products, containing reduced level or no preservative. The present disclosure addresses this and other related needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, provided herein is a personal care composition, which composition comprises: 1) a personal care substance; and 2) a water activity adjusting substance, wherein said personal care composition comprises from about 25% (w/w) water to about 60% (w/w) water, and said personal care composition has a water activity of 0.76 or less.

In another aspect, provided herein is a kit, a package or an article of manufacture, which comprises the above personal care composition. The kit, package or article of manufacture can further comprise separate containers or vials for one or more components and/or instructions for using the kit, package or article of manufacture.

In still another aspect, provided herein are methods of using the above personal care composition, e.g., a method that comprises administering (or applying) the above personal care composition to a subject.

The present disclosure further includes but not limited to the embodiments as below:

Embodiment 1: A personal care composition, which composition comprises:
1) a personal care substance; and
2) a water activity adjusting substance,
wherein said personal care composition comprises from about 25% (w/w) water to about 60% (w/w) water, and said personal care composition has a water activity of 0.76 or less.

Embodiment 2: The personal care composition of embodiment 1, wherein the water activity adjusting substance is:
1) a small molecule, e.g., a water-soluble small molecule or a water-insoluble small molecule;
2) a polymer, e.g., a water-soluble polymer or a water-insoluble polymer; and/or
3) a combination of 1) and 2).

Embodiment 3: The personal care composition of embodiment 2, wherein the water-soluble small molecule is a water-soluble carbohydrate.

Embodiment: 4: The personal care composition of embodiment 3, wherein the water-soluble carbohydrate is a sugar, an oligosaccharide or a polysaccharide.

Embodiment 5: The personal care composition of embodiment 4, wherein the sugar is a monosaccharide, disaccharide or a polyol.

Embodiment 6: The personal care composition of embodiment 5, wherein the monosaccharide is glucose, fructose, galactose or xylose.

Embodiment 7: The personal care composition of embodiment 5, wherein the disaccharide is sucrose, lactose, maltose or trehalose.

Embodiment 8: The personal care composition of embodiment 5, wherein the polyol is a sugar alcohol.

Embodiment 9: The personal care composition of embodiment 8, wherein the sugar alcohol is ethylene glycol, glycerol, erythritol, threitol, arabitol, xylitol, ribitol, mannitol, sorbitol, galactitol, fucitol, iditol, inositol or volemitol.

Embodiment 10: The personal care composition of embodiment 4, wherein the oligosaccharide is a n-linked oligosaccharide or an o-linked oligosaccharide.

Embodiment 11: The personal care composition of embodiment 4, wherein the oligosaccharide is raffinose, stachyose, a fructo-oligosaccharide, or a malto-oligosaccharide, e.g., maltodextrin.

Embodiment 12: The personal care composition of embodiment 4, wherein the polysaccharide is starch, e.g., amylose, amylopectin or a modified starch.

Embodiment 13: The personal care composition of embodiment 4, wherein the polysaccharide is a non-starch polysaccharide, e.g., glycogen, cellulose, hemicellulose, or pectin.

Embodiment 14: The personal care composition of embodiment 1 or 2, wherein the water activity adjusting substance is glucose, fructose, trehalose, sucrose, honey, erythritol, glycerin, propylene glycol, sorbitol, maltitol, isomalt, lactitol, mannitol, 1,3-propanediol, 1,3-butanediol, betaine, alginic acid, trehalose, xylitol, urea, a water-soluble polyol, or a polysaccharide, e.g., dextrin or malt polysaccharide.

Embodiment 15: The personal care composition of embodiment 2, wherein the water-soluble polymer is carboxymethyl cellulose, a modified water-soluble cellulose derivative, starch, a modified starch, glycan, pullulan, hyaluronic acid, beta-glucan, agar, sodium alginate, a water-soluble shell, e.g., carboxymethyl chitin, chitosan or hydroxypropyl chitosan, or a gum, e.g., natto gum, linseed gum, locust bean gum, guar gum, gum arabic, acacia gum, cassia gum, welan gum or xanthan gum.

Embodiment 16: The personal care composition of any of embodiments 1-15, wherein the water activity adjusting substance has a level or concentration from about 0.01% (w/w) to about 75% (w/w).

Embodiment 17: The personal care composition of embodiment 16, wherein the water activity adjusting substance is a small molecule, e.g., a water-soluble small molecule, that has a level or concentration from about 0.01% (w/w) to about 75% (w/w).

Embodiment 18: The personal care composition of embodiment 16, wherein the water activity adjusting substance is a polymer, e.g., a water-soluble polymer, that has a level or concentration from about 0.01% (w/w) to about 30% (w/w).

Embodiment 19: The personal care composition of any of embodiments 1-18, which comprises a single water activity adjusting substance.

Embodiment 20: The personal care composition of any of embodiments 1-18, which comprises multiple water activity adjusting substances.

Embodiment 21: The personal care composition of embodiment 20, wherein the water activity adjusting substances comprises multiple water-soluble small molecules or multiple water-soluble polymers.

Embodiment 22: The personal care composition of embodiment 20, wherein the water activity adjusting substances comprises a water-soluble small molecule and a water-soluble polymer.

Embodiment 23: The personal care composition of any of embodiments 1-22, which comprises from about 25% (w/w) water to about 52% (w/w) water.

Embodiment 24: The personal care composition of any of embodiments 1-23, which has a water activity from about 0.5 to about 0.76.

Embodiment 25: The personal care composition of any of embodiments 1-24, which comprises a preservative content, e.g., an antimicrobial preservative content that is from about one tenth to about one fifth of the preservative content that is needed in a corresponding personal care composition that has a water activity of 0.8 or more.

Embodiment 26: The personal care composition of embodiment 25, wherein the antimicrobial preservative is an acid, an ester, an alcohol, a phenol, a mercurial compound, or a quaternary ammonium compound.

Embodiment 27: The personal care composition of embodiment 26, wherein the acid is benzoic acid, a sorbic acid, or a boric acid.

Embodiment 28: The personal care composition of embodiment 26, wherein the ester is methylparaben, ethylparaben, propylparaben, butylparaben, sodium benzoate, sodium propionate, or potassium sorbate.

Embodiment 29: The personal care composition of embodiment 26, wherein the alcohol is chlorobutanol, benzyl alcohol, or phenyl ethyl alcohol.

Embodiment 30: The personal care composition of embodiment 26, wherein the phenol is phenol, chlorocresol, or o-phenyl phenol.

Embodiment 31: The personal care composition of embodiment 26, wherein the mercurial compound is thiomersal, nitromersol, phenylmercuric nitrate, or phenylmercuric acetate.

Embodiment 32: The personal care composition of embodiment 26, wherein the quaternary ammonium compound is benzalkonium chloride or cetyl pyridinium chloride.

Embodiment 33: The personal care composition of any of embodiments 1-24, which does not comprise a preservative e.g., an antimicrobial preservative, or does not comprise a chemically synthesized preservative e.g., a chemically synthesized antimicrobial preservative.

Embodiment 34: The personal care composition of any of embodiments 1-33, which has a surfactant content of 8% (w/w) or less.

Embodiment 35: The personal care composition of embodiment 34, wherein the surfactant is a detergent, a wetting agent, an emulsifier, a foaming agent, or a dispersant.

Embodiment 36: The personal care composition of embodiment 35, wherein the detergent is an anionic detergent, a cationic detergent or an non-ionic and zwitterionic detergent.

Embodiment 37: The personal care composition of embodiment 35, wherein the foaming agent is sodium laureth sulfate (SLES), sodium dodecyl sulfate (SDS) or ammonium lauryl sulfate (ALS).

Embodiment 38: The personal care composition of any of embodiments 1-33, which does not comprise a surfactant, e.g., a chemically synthesized surfactant.

Embodiment 39: The personal care composition of any of embodiments 1-38, which further comprises a stabilizing substance that reduces or prevents precipitation, sedimentation or crystallization of a component of the personal care composition.

Embodiment 40: The personal care composition of embodiment 39, wherein the stabilizing substance reduces or prevents precipitation, sedimentation or crystallization of the personal care substance, the water activity adjusting substance and/or water of the personal care composition.

Embodiment 41: The personal care composition of embodiment 39 or 40, wherein the stabilizing substance is a substance that enhances solubility of the water activity adjusting substance, e.g., an amide of a carbonic acid such as urea.

Embodiment 42: The personal care composition of any of embodiments 1-41, wherein the personal care substance comprises a natural substance or a substance that is not chemically synthesized.

Embodiment 43: The personal care composition of embodiment 42, wherein the personal care substance comprises a plant extract or a plant extract component.

Embodiment 44: The personal care composition of embodiment 43, wherein the personal care substance comprises an extract or an extract component from aloe vera, rose, calendula, lavender, jojoba, tea tree, chamomile, geranium, basil, rosemary, spanish chestnut, date palm, almond kernel, pomegranate, pine bark, and/or coffeeberry.

Embodiment 45: The personal care composition of embodiment 42, wherein the personal care substance comprises:

a) a prebiotic, or a substance that induces and/or supports growth of microbe that is beneficial to a subject;

b) a probiotic; and/or

c) a postbiotic.

Embodiment 46: The personal care composition of embodiment 45, wherein the postbiotic comprises an intracellular metabolic product of a probiotic, an extracellular metabolic product of a probiotic, or a combination thereof.

Embodiment 47: The personal care composition of embodiment 45 or 46, which comprises at least two of a), b) and c), e.g., a) and b), a) and c), orb and c).

Embodiment 48: The personal care composition of any of embodiments 44-47, wherein the probiotic comprises a bacterium and/or fungus.

Embodiment 49: The personal care composition of embodiment 48, wherein the probiotic comprises a lactic acid bacterium or a nitrifying bacterium.

Embodiment 50: The personal care composition of embodiment 48, wherein the probiotic comprises a species of Aerococcus, Bifidobacterium, Carnobacterium, Enterococcus, Lactobacillus, Lactococcus, Leuconostoc, Oenococcu, Pediococcus, Sporolactobacillus, Streptococcus, Tetragenococcus, Vagococcus or Weissella.

Embodiment 51: The personal care composition of embodiment 50, wherein the species of Bifidobacterium is Bifidobacterium bifidum, Bifidobacterium lactis or Bifidobacterium longum.

Embodiment 52: The personal care composition of embodiment 50, wherein the species of Lactobacillus is Lactobacillus acidophilus, Lactobacillus bulgaricus, Lactobacillus casei, Lactobacillus gasseri, Lactobacillus fermentum, e.g., Lactobacillus fermentum strain having an Accession No. PTA-122195, or Lactobacillus plantarum.

Embodiment 53: The personal care composition of embodiment 50, wherein the species of Enterococcus is Enterococcus faecium.

Embodiment 54: The personal care composition of embodiment 49, wherein the nitrifying bacterium is an ammonia-oxidizing bacterium (AOB) or a nitrite-oxidizing bacterium (NOB).

Embodiment 55: The personal care composition of embodiment 54, wherein the ammonia-oxidizing bacterium is a species of Nitrosomonas, Nitrosococcus or Nitrosospira.

Embodiment 56: The personal care composition of embodiment 54, wherein the nitrite-oxidizing bacterium is a species of Nitrobacter, Nitrospina, Nitrococcus, or Nitrospira.

Embodiment 57: The personal care composition of embodiment 48, wherein the fungus is a species of Saccharomyces.

Embodiment 58: The personal care composition of embodiment 57, wherein the species of Saccharomyces is Saccharomyces boulardii.

Embodiment 59: The personal care composition of embodiment 44, wherein a) comprises a fermentation medium or fermentation medium component.

Embodiment 60: The personal care composition of embodiment 59, wherein the fermentation medium or fermentation medium component comprises a grain-based fermentation medium or fermentation medium component.

Embodiment 61: The personal care composition of embodiment 60, wherein the grain-based fermentation medium or fermentation medium component comprises rice-based or corn-based fermentation medium or fermentation medium component.

Embodiment 62: The personal care composition of any of embodiments 1-61, wherein the personal care substance has a level or concentration from about 0.01% (w/w) to about 75% (w/w).

Embodiment 63: The personal care composition of any of embodiments 1-62, which is configured to have cleansing, protecting, e.g., protecting against sun burn, smoothing, moisturizing, softening, perfuming, beautifying and/or treating function.

Embodiment 64: The personal care composition of any of embodiments 1-63, which is configured to be a skin care, hair care, oral care, shaving and/or grooming, sun care, deodorant and/or antiperspiran, bath and/or body, incontinence, pregnancy and/or fertility, feminine care, eye care, ear, nose and/or throat care, foot care, lip care, or massage and/or relaxation composition or product.

Embodiment 65: The personal care composition of embodiment 64, wherein the hair care composition or product is shampoo and/or conditioner, salon hair care, hair styling, hair coloring, hair and/or scalp treatment composition or product.

Embodiment 66: The personal care composition of embodiment 64, wherein the oral care composition or product is toothpaste, mouthwash and/or rinse, or fresh breath composition or product.

Embodiment 67: The personal care composition of embodiment 64, wherein the shaving and/or grooming composition or product is shave cream, shave gel or bald head care composition or product.

Embodiment 68: The personal care composition of embodiment 64, wherein the sun care composition or product is sunscreen, self-tanner, tan enhancer and/or bronzer, or after sun care composition or product.

Embodiment 69: The personal care composition of embodiment 64, wherein the deodorant and/or antiperspiran composition or product is body spray composition or product.

Embodiment 70: The personal care composition of embodiment 64, wherein the bath and/or body composition or product is body cleansing, body moisturizer, hand moisturizer, exfoliator and/or scrub, body treatment, hand soap, hand sanitizer, or body spray composition or product.

Embodiment 71: The personal care composition of embodiment 64, wherein the incontinence composition or product is cleanser, cream and/or lotion composition or product.

Embodiment 72: The personal care composition of embodiment 64, wherein the feminine care composition or product is intimate cleansing and/or deodorant, or vaginal cream and/or moisturizer composition or product.

Embodiment 73: The personal care composition of embodiment 64, wherein the eye care composition or product is contact lens solution, eye drop, e.g., eye drop for contact lens, allergy and/or itchy eye relief, dry eye relief, or redness relief composition or product.

Embodiment 74: The personal care composition of embodiment 64, wherein the ear, nose and/or throat care composition or product is earache and/or infection relief, nasal spray, or sinus medicine composition or product.

Embodiment 75: The personal care composition of embodiment 64, wherein the foot care composition or product is corn, callus, blister and/or bunion relief, foot cream and/or treatment, diabetic foot care, athlete's foot and/or antifungal, or odor control composition or product.

Embodiment 76: The personal care composition of embodiment 64, wherein the lip care composition or product is lip balm, or lip and/or cold sore treatment composition or product.

Embodiment 77: The personal care composition of embodiment 64, wherein the massage and/or relaxation composition or product is massage lotion and/or cream composition or product.

Embodiment 78: The personal care composition of embodiment 64, wherein the skin care composition or product is skin cleanser, skin moisturizer, skin lotion, skin essence, a makeup remover, or a topical medicine.

Embodiment 79: The personal care composition of any of embodiments 1-63, which is configured to be a cosmetic or make-up composition or product.

Embodiment 80: The personal care composition of embodiment 79, wherein the cosmetic or make-up composition or product is lipstick, lip gloss, lip liner, lip balm, lip stain, lip conditioner, lip primer, lip booster, lip butter, primer, concealer, foundation, rouge, highlighter, bronzer, mascara, or setting spray composition or product.

Embodiment 81: The personal care composition of embodiment 79, wherein the cosmetic or make-up composition or product is a cleanser, foaming wash, toner, facial mask, exfoliation, or moisturizer.

Embodiment 82: The personal care composition of any of embodiments 1-81, which comprises a single personal care substance.

Embodiment 83: The personal care composition of any of embodiments 1-81, which comprises multiple personal care substances.

Embodiment 84: The personal care composition of embodiment 83, which comprises a plant extract or a plant extract component, and at least one of the following:

a) a prebiotic, or a substance that induces and/or supports growth of microbe that is beneficial to a subject;

b) a probiotic; and/or
c) a postbiotic.

Embodiment 85: The personal care composition of embodiment 84, which comprises a plant extract or a plant extract component, and a prebiotic or a postbiotic.

Embodiment 86: The personal care composition of any of embodiments 1-85, which does not comprise a preservative content, e.g., an antimicrobial preservative, and does not comprise a surfactant.

Embodiment 87: The personal care composition of embodiment 86, which does not comprise an antimicrobial preservative, and does not comprise a surfactant.

Embodiment 88: The personal care composition of embodiment 86 or 87, which further comprises a stabilizing substance that reduces or prevents precipitation, sedimentation or crystallization of a component of the personal care composition.

Embodiment 89: The personal care composition of any of embodiments 1-88, which is self-preserving.

Embodiment 90: The personal care composition of embodiment 89, which is configured to reduce or inhibit viral or microbial growth prior to use.

Embodiment 91: The personal care composition of embodiment 90, which is configured to reduce or inhibit growth of a virus, bacterium, fungus and/or parasite prior to use.

Embodiment 92: The personal care composition of embodiment 91, which is configured to reduce or inhibit growth of a undesirable or pathogenic virus, bacterium, fungus and/or parasite, or a treatment resistant virus, bacterium, fungus and/or parasite, e.g., antibiotic-resistant bacterium, prior to use.

Embodiment 93: The personal care composition of embodiment 92, wherein the virus is Herpes simplex virus.

Embodiment 94: The personal care composition of embodiment 92, wherein the bacterium is Staphylococcus aureus, e.g., methicillin-resistant Staphylococcus aureus (MRSA), or group A β-hemolytic streptococci.

Embodiment 95: The personal care composition of embodiment 92, wherein the fungus is Trichophyton rubrurn.

Embodiment 96: The personal care composition of any of embodiments 90-95, which is configured to reduce or inhibit viral or microbial growth during manufacturing, transportation and/or storage.

Embodiment 97: The personal care composition of any of embodiments 1-96, which does dot comprise any substance that is chemically synthesized.

Embodiment 98: The personal care composition of any of embodiments 1-97, which is configured, when applied to a subject, to have reduced or no damage to the subject and/or the subject's cell, tissue organ.

Embodiment 99: The personal care composition of embodiment 98, which is configured, when applied to a subject, to have reduced or no damage to subject's skin or an external cavity.

Embodiment 100: The personal care composition of any of embodiments 1-99, which is configured, when applied to a subject, to have reduced or no damage to the subject's microbiorne (or microbiota).

Embodiment 101: The personal care composition of embodiment 100, which is configured, when applied to a subject, to have reduced or no damage to the subject's microbiome in or on the subject's skin or an external cavity.

Embodiment 102: The personal care composition of any of embodiments 1-100, which is in the form of a liquid, a spray, a cream, an ointment, a liniment (balm), a paste or a film.

Embodiment 103: The personal care composition of any of embodiments 1-102, which is configured to be a concentrate that needs to be diluted for use, e.g., needs to be diluted using water or an aqueous liquid for use.

Embodiment 104: A kit, a package or an article of manufacture, which comprises a personal care composition of any of embodiments 1-103.

Embodiment 105: The kit, package or article of manufacture of embodiment 104, which comprises a single personal care composition of any of embodiments 1-103.

Embodiment 106: The kit, package or article of manufacture of embodiment 104, which comprises multiple personal care compositions of any of embodiments 1-103.

Embodiment 107: The kit, package or article of manufacture of any of embodiments 104-106, which further comprise an instruction for using the personal care composition of any of embodiments 1-103.

Embodiment 108: A method, which comprises administering (or applying) a personal care composition of any of embodiments 1-103 to a subject.

Embodiment 109: The method embodiment 108, which further comprises:
1) diluting the personal care composition from about 50% to about 5 folds;
2) diluting the personal care composition to a degree so that the administered (or applied) personal care composition has a water activity of about 8 or higher; and/or
3) diluting the personal care composition to a degree so that the administered (or applied) personal care composition has reduced or no damage to the subject's microbiome (or microbiota).

Embodiment 110: The method embodiment 108, which comprises administering (or applying) the personal care composition to the subject's skin or an external cavity, and wherein the administered (or applied) personal care composition has reduced or no damage to the subject's microbiome (or microbiota).

Embodiment 111: The method of embodiment 109 or 110, wherein the personal care composition is diluted before, during or after administering (or applying) the personal care composition to the subject.

Embodiment 112: The method of any of embodiments 109-111, which comprises diluting the personal care composition into a diluted personal care composition, and administering (or applying) the diluted personal care composition to a subject.

Embodiment 113: The method of any of embodiments 109-111, which comprises administering (or applying) an exogeneous aqueous liquid to a target site of a subject, and administering (or applying) the personal care composition to the target site of the subject, wherein the exogeneous aqueous liquid provides the dilution for the administered (or applied) personal care composition.

Embodiment 114: The method of any of embodiments 109-111, which comprises administering (or applying) the personal care composition to a target site of a subject that has endogenous liquid, wherein the endogenous liquid provides the dilution for the administered (or applied) personal care composition.

Embodiment 115: The method of any of embodiments 108-114, which is used for cleansing, protecting, smoothing, moisturizing, softening, perfuming, beautifying and/or treating a subject, or a target area, cell, tissue or organ of a subject.

Embodiment 116: The method of any of embodiments 108-115, wherein the subject is animal, e.g., a pet.

Embodiment 117: The method of any of embodiments 108-115, wherein the subject is a human.

Embodiment 118: The method embodiment 117, wherein the human is an infant, a child, a teenager, an adult, a senior, a male, a female, a pregnant woman, a healthy human, or a patient

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

### A. DEFINITIONS

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.” As used herein, the singular forms “a,” “an,” and “the” include the plural reference unless the context clearly dictates otherwise.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “average” as used herein refers to either a mean or a median, or any value used to approximate the mean or the median, unless the context clearly indicates otherwise.

As used herein the term “sample” refers to anything which may contain a target molecule for which analysis is desired, including a biological sample. As used herein, a “biological sample” can refer to any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine, sweat, semen, stool, sputum, tears, mucus, amniotic fluid or the like, an effusion, a bone marrow sample, ascitic fluid, pelvic wash fluid, pleural fluid, spinal fluid, lymph, ocular fluid, extract of nasal, throat or genital swab, cell suspension from digested tissue, or extract of fecal material, and tissue and organ samples from animals and plants and processed samples derived therefrom.

As used herein, “disease or disorder” refers to a pathological condition in an organism resulting from, e.g., infection or genetic defect, and characterized by identifiable symptoms.

As used herein, a “preservative” refers to a substance or a chemical that is added to a personal care composition, e.g., skin care products or cosmetics, to reduce or prevent damage, decomposition or other undesirable effects by microbial growth and/or metabolism, or by an undesirable chemical reaction. As used herein, an “antimicrobial preservative” refers to a substance or a chemical that is added to a personal care composition, e.g., skin care products or cosmetics, to reduce or prevent damage, decomposition or other undesirable effects by microbial growth and/or metabolism.

As used herein, a “surfactant” refers to a substance or a chemical that lowers the surface tension (or interfacial tension) between two liquids, between a gas and a liquid, or between a liquid and a solid. A surfactant is usually an organic compound that is amphiphilic, meaning it contains both a hydrophobic group and a hydrophilic group. Therefore, a surfactant contains both a water-insoluble (or oil-soluble) component and a water-soluble component. A surfactants may diffuse in water and absorbs at interface between air and water or at the interface between oil and water, in the case where water is mixed with oil. The water-insoluble hydrophobic group may extend out of the bulk water phase, into the air or into the oil phase, while the water-soluble group may remain in the water phase.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, a subject refers to an animal, a non-human mammal or a human. As used herein, “animals” include a pet, a farm animal, an economic animal, a sport animal and an experimental animal, such as a cat, a dog, a horse, a cow, an ox, a pig, a donkey, a sheep, a lamb, a goat, a mouse, a rabbit, a chicken, a duck, a goose, a primate, including a monkey and a chimpanzee.

Other objects, advantages and features of the present invention will become apparent from the following specification taken in conjunction with the accompanying drawings.

### B. PERSONAL CARE COMPOSITIONS AND RELATED KITS, PACKAGES AND ARTICLES OF MANUFACTURE

In one aspect, provided herein is a personal care composition, which composition comprises: 1) a personal care substance; and 2) a water activity adjusting substance, wherein said personal care composition comprises from about 25% (w/w) water to about 60% (w/w) water, and said personal care composition has a water activity of 0.76 or less.

The present personal care composition can comprise any suitable water activity adjusting substance. For example, the present personal care composition can comprise a small molecule water activity adjusting substance, e.g., a water-soluble small molecule or a water-insoluble small molecule water activity adjusting substance. In another example, the present personal care composition can comprise a polymer water activity adjusting substance, e.g., a water-soluble polymer or a water-insoluble polymer water activity adjusting substance. In another example, the present personal care composition can comprise a combination of a small molecule water activity adjusting substance and a polymer water activity adjusting substance.

The present personal care composition can comprise any suitable small molecule water activity adjusting substance. For example, the water activity adjusting substance in the present personal care composition can comprise a water-soluble carbohydrate. Any suitable water-soluble carbohydrate can be used. For example, the water-soluble carbohydrate can be a sugar, an oligosaccharide or a polysaccharide.

The water activity adjusting substance in the present personal care composition can comprise any suitable sugar. For example, the sugar can be a monosaccharide, disaccharide or a polyol. In some embodiments, the monosaccharide is glucose, fructose, galactose or xylose. In some embodiments, the disaccharide is sucrose, lactose, maltose or trehalose. In some embodiments, the polyol is a sugar alcohol. Exemplary sugar alcohols include ethylene glycol, glycerol, erythritol, threitol, arabitol, xylitol, ribitol, mannitol, sorbitol, galactitol, fucitol, iditol, inositol or volemitol.

The water activity adjusting substance in the present personal care composition can comprise any suitable oligosaccharide. For example, the oligosaccharide can be a n-linked oligosaccharide or an o-linked oligosaccharide. In some embodiments, the oligosaccharide is raffinose, stachyose, a fructo-oligosaccharide, or a malto-oligosaccharide, e.g., maltodextrin.

The water activity adjusting substance in the present personal care composition can comprise any suitable polysaccharide. For example, the polysaccharide can be a starch, e.g., amylose, amylopectin or a modified starch. In another example, the polysaccharide can be a non-starch polysaccharide, e.g., glycogen, cellulose, hemicellulose, or pectin.

In some embodiments, the water activity adjusting substance in the present personal care composition can comprise glucose, fructose, trehalose, sucrose, honey, erythritol, glycerin, propylene glycol, sorbitol, maltitol, isomalt, lactitol, mannitol, 1,3-propanediol, 1,3-butanediol, betaine, alginic acid, trehalose, xylitol, urea, a water-soluble polyol, or a polysaccharide, e.g., dextrin or malt polysaccharide.

The water activity adjusting substance in the present personal care composition can comprise any suitable polymer. For example, the water-soluble polymer can comprise carboxymethyl cellulose, a modified water-soluble cellulose derivative, starch, a modified starch, glycan, pullulan, hyaluronic acid, beta-glucan, agar, sodium alginate, a water-soluble shell, e.g., carboxymethyl chitin, chitosan or hydroxypropyl chitosan, or a gum, e.g., natto gum, linseed gum, locust bean gum, guar gum, gum arabic, acacia gum, cassia gum, welan gum or xanthan gum.

The water activity adjusting substance in the present personal care composition can have any suitable level or concentration. For example, the water activity adjusting substance in the present personal care composition can have a level or concentration from about 0.01% (w/w) to about 75% (w/w), e.g., at about 0.01% (w/w), 0.05% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% 20% (w/w), 30% (w/w), 40% (w/w), 50% (w/w), 60% (w/w), 70% (w/w), 75% (w/w), or any sub-range thereof.

The small molecule water activity adjusting substance, e.g., a water-soluble small molecule, in the present personal care composition can have any suitable level or concentration. For example, the small molecule water activity adjusting substance, e.g., a water-soluble small molecule, in the present personal care composition can have a level or concentration from about 0.01% (w/w) to about 75% (w/w), e.g., at about 0.01% (w/w), 0.05% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% 20% (w/w), 30% (w/w), 40% (w/w), 50% (w/w), 60% (w/w), 70% (w/w), 75% (w/w), or any sub-range thereof.

The polymer water activity adjusting substance, e.g., a water-soluble polymer, in the present personal care composition can have any suitable level or concentration. For example, the polymer water activity adjusting substance, e.g., a water-soluble polymer, in the present personal care composition can have a level or concentration from about 0.01% (w/w) to about 75% (w/w), e.g., at about 0.01% (w/w), 0.05% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 20% (w/w), 30% (w/w), 40% (w/w), 50% (w/w), 60% (w/w), 70% (w/w), 75% (w/w), or any sub-range thereof.

The present personal care composition can comprise any suitable number and/or types of water activity adjusting substance(s). For example, the present personal care composition can comprise a single water activity adjusting substance. In another example, the present personal care composition can comprise multiple number and/or types water activity adjusting substances, e.g., 2, 3, 4, 5, 6, 7, 8, 9, 10, 11, 12, 13, 14, 15, 16, 17, 18, 19, 20, 25, 30 or more water activity adjusting substances, which can be the same type or different types of substances.

In some embodiments, the present personal care composition can comprise multiple number and/or types small molecule water activity adjusting substances, e.g., 2, 3, 4, 5, 6, 7, 8, 9, 10, 11, 12, 13, 14, 15, 16, 17, 18, 19, 20, 25, 30 or more small molecule water activity adjusting substances, which can be the same type or different types of substances. The small molecule water activity adjusting substances can be water-soluble small molecules and/or water-insoluble small molecules.

In some embodiments, the present personal care composition can comprise multiple number and/or types polymer water activity adjusting substances, e.g., 2, 3, 4, 5, 6, 7, 8, 9, 10, 11, 12, 13, 14, 15, 16, 17, 18, 19, 20, 25, 30 or more polymer water activity adjusting substances, which can be the same type or different types of substances. The polymer water activity adjusting substances can be water-soluble polymers and/or water-insoluble polymers.

The present personal care can comprise both small molecule water activity adjusting substance(s) and polymer water activity adjusting substance(s). In some embodiments, the present personal care composition can comprise a single small molecule water activity adjusting substance and a single polymer water activity adjusting substance. In other embodiments, the present personal care composition can comprise a single small molecule water activity adjusting substance and multiple number and/or types polymer water activity adjusting substances. In other embodiments, the present personal care composition can comprise multiple number and/or types small molecule water activity adjusting substances and a single polymer water activity adjusting substance. In still other embodiments, the present personal care composition can comprise multiple number and/or types small molecule water activity adjusting substances and multiple number and/or types polymer water activity adjusting substances. The small molecule water activity adjusting substance(s) can be water-soluble small molecule(s) and/or water-insoluble small molecule(s). The polymer water activity adjusting substances can be water-soluble polymer(s) and/or water-insoluble polymer(s).

The present personal care composition can have any suitable level or concentration of water. For example, the present personal care composition can have a water level or concentration from about 25% (w/w) to about 60% (w/w), e.g., at about 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), or any sub-range thereof. In some embodiments, the present personal care composition comprises from about 25% (w/w) water to about 52% (w/w) water, from about 30% (w/w) water to about 50% (w/w) water, from about 35% (w/w) water to about 50% (w/w) water, from about 30% (w/w) water to about 45% (w/w) water, from about 30% (w/w) water to about 40% (w/w) water, or any sub-range thereof.

The present personal care composition can have any suitable level of water activity. For example, present personal care composition can have a water activity from about 0.4 to about 0.76, e.g., at about 0.4, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, or any sub-range thereof.

The present personal care composition can have a reduced level or no preservative. For example, the present personal care composition can have a reduced level or no antimicrobial preservative. In some embodiments, the present personal care composition can comprise a preservative content, e.g., an antimicrobial preservative content, that is from about one tenth to about one fifth of the preservative content that is needed in a corresponding personal care composition that has a water activity of 0.8 or more. In other embodiments, the present personal care composition can comprise a preservative content, e.g., an antimicrobial preservative content, that is about 10% (w/w) or less, e.g., at about 0.001% (w/w), 0.002% (w/w), 0.003% (w/w), 0.004% (w/w), 0.005% (w/w), 0.006% (w/w), 0.007% (w/w), 0.008% (w/w), 0.009% (w/w), 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), or any sub-range thereof.

The present personal care composition can have a reduced level of any suitable type of preservative or antimicrobial preservative. For example, the preservative or antimicrobial preservative can be an acid, an ester, an alcohol, a phenol, a mercurial compound, or a quaternary ammonium compound. In some embodiments, the acid is benzoic acid, a sorbic acid, or a boric acid. In some embodiments, the ester is methylparaben, ethylparaben, propylparaben, butylparaben, sodium benzoate, sodium propionate, or potassium sorbate. In some embodiments, the alcohol is chlorobutanol, benzyl alcohol, or phenyl ethyl alcohol. In some embodiments, the phenol or phenol derivative is phenol, chlorocresol, or o-phenyl phenol. In some embodiments, the mercurial compound is thiomersal, nitromersol, phenylmercuric nitrate, or phenylmercuric acetate. In some embodiments, the quaternary ammonium compound is benzalkonium chloride or cetyl pyridinium chloride.

The preservative or antimicrobial preservative can have any suitable E number, i.e., codes for substances that are permitted to be used as food additives for use within the European Union (EU). In some embodiments, the preservative or antimicrobial preservative have E number of E200-E300, e.g., sorbic acid, sodium sorbate, and sorbates. In some embodiments, the preservative or antimicrobial preservative have E number of E210-E213, e.g., benzoic acid, sodium benzoate, and benzoates. In some embodiments, the preservative or antimicrobial preservative have E number of E214-E219, e.g., hydroxybenzoic acid and derivatives. In some embodiments, the preservative or antimicrobial preservative have E number of E220-E227, e.g., sulfur dioxide and sulfites. In some embodiments, the preservative or antimicrobial preservative have E number of E249-E250, e.g., nitrite. In some embodiments, the preservative or antimicrobial preservative have E number of E251-E252, e.g., nitrate. In some embodiments, the preservative or antimicrobial preservative have E number of E270, e.g., lactic acid. In some embodiments, the preservative or antimicrobial preservative have E number of E280-E283, e.g., propionic acid and sodium propanoate.

In some embodiments, the present personal care composition does not comprise a preservative, or does not comprise an antimicrobial preservative. In some embodiments, the present personal care composition does not comprise a chemically synthesized preservative, or does not comprise a chemically synthesized antimicrobial preservative. In some embodiments, the present personal care composition does not comprise a natural preservative, or does not comprise a natural antimicrobial preservative.

The present personal care composition can have a reduced level or no surfactant. For example, the present personal care composition can have a surfactant content of about 8% (w/w) or less, e.g., at about 0.001% (w/w), 0.002% (w/w), 0.003% (w/w), 0.004% (w/w), 0.005% (w/w), 0.006% (w/w), 0.007% (w/w), 0.008% (w/w), 0.009% (w/w), 0.01% (w/w), 0.02% (w/w), 0.03% (w/w), 0.04% (w/w), 0.05% (w/w), 0.06% (w/w), 0.07% (w/w), 0.08% (w/w), 0.09% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), or any sub-range thereof.

The present personal care composition can have a reduced level of any suitable type of surfactant. For example, the surfactant can be a detergent, a wetting agent, an emulsifier, a foaming agent, or a dispersant. In some embodiments, the detergent is an anionic detergent, a cationic detergent or an non-ionic or zwitterionic detergent. In some embodiments, the foaming agent is sodium laureth sulfate (SLES), sodium dodecyl sulfate (SDS) or ammonium lauryl sulfate (ALS).

In some embodiments, the present personal care composition does not comprise a chemically synthesized surfactant. In some embodiments, the present personal care composition does not comprise a natural surfactant. In some embodiments, the present personal care composition does not comprise a surfactant.

The present personal care composition can further comprise a stabilizing substance that reduces or prevents precipitation, sedimentation or crystallization of a component of the personal care composition. Any suitable stabilizing substance can be used. In some embodiments, the stabilizing substance reduces or prevents precipitation, sedimentation or crystallization of the personal care substance, the water activity adjusting substance and/or water of the personal care composition. In some embodiments, the stabilizing substance is a substance that enhances solubility of the water activity adjusting substance, e.g., an amide of a carbonic acid such as urea.

The present personal care composition can comprise any suitable personal care substance. The type, number and/or level of the personal care substance, at least partially, is determined by the intended use(s) or application(s) of the present personal care composition. The personal care substance in the present personal care composition can comprise a chemical substance, a natural substance, a substance that is not chemically synthesized, or a combination thereof. Preferably, the personal care substance in the present personal care composition comprises a natural substance, a substance that is not chemically synthesized, or a combination thereof.

In some embodiments, the personal care substance in the present personal care composition can comprise a plant extract or a plant extract component. The plant extract or plant extract component can be obtained or derived from any suitable plant(s). For example, the personal care substance in the present personal care composition can comprise an extract or an extract component from aloe vera, rose, calendula, lavender, jojoba, tea tree, chamomile, geranium, basil, rosemary, spanish chestnut, date palm, almond kernel, pomegranate, pine bark, and/or coffeeberry. In another, the personal care substance in the present personal care composition can comprise Chrysanthemum sibiricum extract, Gleditsia japonica fruit extract, Rehmannia glutinosa root extract, Thuja orientalis leaf extract, Sophora angustifolia root extract, moms alba bark extract, acorns calamus root extract, Vitex trifolia fruit extract, Eclipta prostrata extract, Cnidium officinale root extract, Artemisia vulgaris extract, Panax ginseng root extract, Camellia sinensis seed extract, Crinum asiaticum extract, Capsicum annuum fruit extract, or Portulaca oleracea extract.

In some embodiments, the personal care substance in the present personal care composition can comprise: 1) a prebiotic, or a substance that induces and/or supports growth of microbe that is beneficial to a subject; 2) a probiotic; and/or 3) a postbiotic. The personal care substance in the present personal care composition can comprise 1), 2) or 3) alone. The personal care substance in the present personal care composition can also comprise some combination of 1), 2) or 3). For example, the personal care substance in the present personal care composition can comprise at least two of 1), 2) and 3), e.g., 1) and 2), 1) and 3), or 2) and 3). In another example, the personal care substance in the present personal care composition can comprise 1), 2) and 3).

The personal care substance in the present personal care composition can comprise any suitable type of postbiotic. For example, the postbiotic can comprise an intracellular metabolic product of a probiotic, an extracellular metabolic product of a probiotic, or a combination thereof.

The personal care substance in the present personal care composition can comprise any suitable type of probiotic. For example, the probiotic can comprise a bacterium and/or fungus. The probiotic can comprise a lactic acid bacterium or a nitrifying bacterium. For example, the probiotic can comprise a species of Aerococcus, Bifidobacterium, Carnobacterium, Enterococcus, Lactobacillus, Lactococcus, Leuconostoc, Oenococcu, Pediococcus, Sporolactobacillus, Streptococcus, Tetragenococcus, Vagococcus or Weissella.

In some embodiments, the species of Bifidobacterium is Bifidobacterium bifidum, Bifidobacterium lactis or Bifidobacterium longum. In some embodiments, the species of Lactobacillus is Lactobacillus acidophilus, Lactobacillus bulgaricus, Lactobacillus casei, Lactobacillus gasseri, Lactobacillus fermentum, e.g., Lactobacillus fermentum strain having an Accession No. PTA-122195 (see, e.g., U.S. Pat. No. 9,706,778), or Lactobacillus plantarum. In some embodiments, the species of Enterococcus is Enterococcus faecium.

In some embodiments, the nitrifying bacterium is an ammonia-oxidizing bacterium (AOB) or a nitrite-oxidizing bacterium (NOB). The ammonia-oxidizing bacterium can be a species of Nitrosomonas, Nitrosococcus or Nitrosospira. The nitrite-oxidizing bacterium can be a species of Nitrobacter, Nitrospina, Nitrococcus, or Nitrospira.

In some embodiments, the fungus is a species of Saccharomyces, e.g., Saccharomyces boulardii.

The personal care substance in the present personal care composition can comprise a fermentation medium or fermentation medium component. In some embodiments, the fermentation medium or fermentation medium component can comprise a grain-based fermentation medium or fermentation medium component, e.g., rice-based or corn-based fermentation medium or fermentation medium component.

The personal care substance in the present personal care composition can have any suitable level or concentration. For example, the personal care substance in the present personal care composition can have a level or concentration from about 0.01% (w/w) to about 75% (w/w), e.g., at about 0.01% (w/w), 0.05% (w/w), 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 20% (w/w), 30% (w/w), 40% (w/w), 50% (w/w), 60% (w/w), 70% (w/w), 75% (w/w), or any sub-range thereof.

The present personal care composition can be configured to have any suitable function. For example, the present personal care composition can be configured to have cleansing, protecting, e.g., protecting against sun burn, smoothing, moisturizing, softening, perfuming, beautifying and/or treating function.

The present personal care composition can be configured to be any suitable composition or product. For example, the present personal care composition can be configured to be a skin care, hair care, oral care, shaving and/or grooming, sun care, deodorant and/or antiperspiran, bath and/or body, incontinence, pregnancy and/or fertility, feminine care, eye care, ear, nose and/or throat care, foot care, lip care, or massage and/or relaxation composition or product.

The present personal care composition can be configured to be any suitable hair care composition or product. Exemplary hair care composition or product can be shampoo and/or conditioner, salon hair care, hair styling, hair coloring, hair and/or scalp treatment composition or product.

The present personal care composition can be configured to be any suitable oral care composition or product. Exemplary oral care composition or product can be toothpaste, mouthwash and/or rinse, or fresh breath composition or product.

The present personal care composition can be configured to be any suitable shaving and/or grooming composition or product. Exemplary shaving and/or grooming composition or product can be shave cream, shave gel or bald head care composition or product.

The present personal care composition can be configured to be any suitable sun care composition or product. Exemplary sun care composition or product can be sunscreen, self-tanner, tan enhancer and/or bronzer, or after sun care composition or product.

The present personal care composition can be configured to be any suitable deodorant and/or antiperspiran composition or product. Exemplary deodorant and/or antiperspiran composition or product can be body spray composition or product.

The present personal care composition can be configured to be any suitable bath and/or body composition or product. Exemplary bath and/or body composition or product can be body cleansing, body moisturizer, hand moisturizer, exfoliator and/or scrub, body treatment, hand soap, hand sanitizer, or body spray composition or product.

The present personal care composition can be configured to be any suitable incontinence composition or product. Exemplary incontinence composition or product can be cleanser, cream and/or lotion composition or product.

The present personal care composition can be configured to be any suitable feminine care composition or product. Exemplary feminine care composition or product can be intimate cleansing and/or deodorant, or vaginal cream and/or moisturizer composition or product.

The present personal care composition can be configured to be any suitable eye care composition or product. Exemplary eye care composition or product can be contact lens solution, eye drop, e.g., eye drop for contact lens, allergy and/or itchy eye relief, dry eye relief, or redness relief composition or product.

The present personal care composition can be configured to be any suitable ear, nose and/or throat care composition or product. Exemplary ear, nose and/or throat care composition or product can be earache and/or infection relief, nasal spray, or sinus medicine composition or product.

The present personal care composition can be configured to be any suitable foot care composition or product. Exemplary foot care composition or product can be corn, callus, blister and/or bunion relief, foot cream and/or treatment, diabetic foot care, athlete's foot and/or antifungal, or odor control composition or product.

The present personal care composition can be configured to be any suitable lip care composition or product. Exemplary lip care composition or product can be lip balm, or lip and/or cold sore treatment composition or product.

The present personal care composition can be configured to be any suitable massage and/or relaxation composition or product. Exemplary massage and/or relaxation composition or product can be massage lotion and/or cream composition or product.

The present personal care composition can be configured to be any suitable skin care composition or product. Exemplary skin care composition or product can be skin cleanser, skin moisturizer, skin lotion, skin essence, a makeup remover, or a topical medicine.

The present personal care composition can be configured to be a cosmetic or make-up composition or product. Exemplary cosmetic or make-up composition or product can be lipstick, lip gloss, lip liner, lip balm, lip stain, lip conditioner, lip primer, lip booster, lip butter, primer, concealer, foundation, rouge, highlighter, bronzer, mascara, or setting spray composition or product. In some embodiments, the cosmetic or make-up composition or product is a cleanser, foaming wash, toner, facial mask, exfoliation, or moisturizer.

The present personal care composition can comprise any suitable number and/or type of personal care substance(s). In some embodiments, the present personal care composition can comprise a single personal care substance. In other embodiments, the present personal care composition can comprise multiple number and/or types of personal care substances. For example, the present personal care composition can comprise multiple number of the same type of personal care substances. In another example, the present personal care composition can comprise multiple number of different types of personal care substances.

In some embodiments, the present personal care composition can comprise a plant extract or a plant extract component, and at least one of the following: 1) a prebiotic, or a substance that induces and/or supports growth of microbe that is beneficial to a subject; 2) a probiotic; and/or 3) a postbiotic. In other embodiments, the present personal care composition can comprise a plant extract or a plant extract component, and a probiotic. In still other embodiments, the present personal care composition can comprise a plant extract or a plant extract component, and a prebiotic or a postbiotic. In yet other embodiments, the present personal care composition can comprise a plant extract or a plant extract component, a prebiotic and a postbiotic. In yet other embodiments, the present personal care composition can comprise a plant extract or a plant extract component, a prebiotic, a probiotic and a postbiotic.

In some embodiments, the present personal care composition does not comprise a preservative (whether chemically synthesized preservative or natural preservative), e.g., an antimicrobial preservative (whether chemically synthesized antimicrobial preservative or natural antimicrobial preservative), and does not comprise a surfactant (whether chemically synthesized surfactant or natural surfactant). In some embodiments, the present personal care composition does not comprise an antimicrobial preservative, and does not comprise a surfactant. In some embodiments, the present personal care composition does not comprise a chemically synthesized antimicrobial preservative, and does not comprise a chemically synthesized surfactant. The present personal care composition can further comprise a stabilizing substance that reduces or prevents precipitation, sedimentation or crystallization of a component of the personal care composition.

The present personal care composition can be self-preserving, e.g., self-preserving against microbial infection or growth. For example, the present personal care composition can be configured to reduce or inhibit viral or microbial growth prior to use. In another example, the present personal care composition can be configured to reduce or inhibit growth of a virus, bacterium, fungus and/or parasite prior to use.

In some embodiments, the present personal care composition can be configured to reduce or inhibit growth of a undesirable or pathogenic virus, bacterium, fungus and/or parasite, or a treatment resistant virus, bacterium, fungus and/or parasite, e.g., antibiotic-resistant bacterium, prior to use. Exemplary virus can be Herpes simplex virus. Exemplary bacterium can be Staphylococcus aureus, e.g., methicillin-resistant Staphylococcus aureus (MRSA), or group A β-hemolytic streptococci. Exemplary fungus can be Trichophyton rubrum.

In some embodiments, the present personal care composition can be configured to reduce or inhibit viral or microbial growth during manufacturing, transportation and/or storage.

In some embodiments, the present personal care composition does dot comprise any substance that is chemically synthesized.

In some embodiments, the present personal care composition can be configured, when applied to a subject, to have reduced or no damage to the subject and/or the subject's cell, tissue organ. For example, the present personal care composition can be configured, when applied to a subject, to have reduced or no damage to subject's skin or an external cavity.

In some embodiments, the present personal care composition can be configured, when applied to a subject, to have reduced or no damage to the subject's microbiome (or microbiota). For example, the present personal care composition can be configured, when applied to a subject, to have reduced or no damage to the subject's microbiome in or on the subject's skin or an external cavity.

The present personal care composition can be configured in any suitable form. For example, the present personal care composition can be configured in the form of a liquid, a spray, a cream, an ointment, a liniment (balm), a paste, a film, or a combination thereof.

In some embodiments, the present personal care composition can be configured to be a concentrate that needs to be diluted for use. For example, the present personal care composition can be configured to need dilution using water or an aqueous liquid for use. The present personal care composition can be diluted to any suitable degree or fold for use. For example, the present personal care composition can be diluted from about 50% to about 5 folds for use, e.g., diluted for about 50%, 1 fold, 2 folds, 3 folds, 4 folds, or 5 folds, for use.

In another aspect, provided herein is a kit, a package or an article of manufacture, which comprises any of the above personal care composition, a combination thereof.

The present kit, a package or an article of manufacture can comprise any suitable number and/or type of the above personal care composition, a combination thereof. In some embodiments, the present kit, package or article of manufacture comprises a single personal care composition as described above. In other embodiments, the present kit, package or article of manufacture comprises multiple number and/or type of the above personal care compositions. For example, the present kit, package or article of manufacture comprises multiple number of the above personal care compositions. In another example, the present kit, package or article of manufacture comprises multiple types of the above personal care compositions. In still another example, the present kit, package or article of manufacture comprises multiple number and multiple types of the above personal care compositions.

In some embodiments, the present kit, package or article of manufacture further comprises an instruction for using the above personal care composition(s).

In some embodiments, one container contains the present personal care composition and another container contains a dilution liquid, e.g., water. In use, the present personal care composition and the dilution liquid are mixed, e.g., by pushing the containers, to form a diluted personal care composition, which can be delivered through a delivering tube. In some embodiments, at least one or both of the containers do not comprise a preservative e.g., an antimicrobial preservative. In some embodiments, both of the containers and the delivering tube do not comprise a preservative e.g., an antimicrobial preservative.

### C. METHODS OF ADMINISTERING OR APPLYING A PERSONAL CARE COMPOSITION

In still another aspect, provided herein is a method, which method comprises administering (or applying) a personal care composition as described in the above Section B. to a subject.

The present method can further comprise diluting the personal care composition, before, during and/or after administering (or applying) to a subject.

The present personal care composition can be diluted to any suitable degree. In some embodiments, the present personal care composition can be diluted from about 50% to about 5 folds for use, e.g., diluted for about 50%, 1 fold, 2 folds, 3 folds, 4 folds, or 5 folds, for use. In other embodiments, the present personal care composition can be diluted to a degree so that the administered (or applied) personal care composition has a water activity of about 8 or higher, e.g., about 8.0, 8.1, 8.2, 8.3, 8.4, 8.5, 8.6, 8.7, 8.8, 8.9, 9.0, 9.1, 9.2, 9.3, 9.4, 9.5 or higher. In still other embodiments, the present personal care composition can be diluted to a degree so that the administered (or applied) personal care composition has reduced or no damage to the subject's microbiome (or microbiota).

In some embodiments, the present method comprises administering (or applying) the personal care composition to the subject's skin or an external cavity, and wherein the administered (or applied) personal care composition has reduced or no damage to microbiome (or microbiota) of the subject's skin or external cavity.

The personal care composition can be diluted at or during any suitable timeframe. For example, the personal care composition can be diluted before, during or after administering (or applying) the personal care composition to the subject.

The personal care composition can be diluted in any suitable manner. For example, the present method can comprise diluting the personal care composition into a diluted personal care composition, and administering (or applying) the diluted personal care composition to a subject. In another example, the present method can comprise administering (or applying) an exogeneous aqueous liquid to a target site of a subject, and administering (or applying) the personal care composition to the target site of the subject, wherein the exogeneous aqueous liquid provides the dilution for the administered (or applied) personal care composition. In still another example, the present method can comprise administering (or applying) the personal care composition to a target site of a subject that has endogenous liquid, e.g., sweat, wherein the endogenous liquid provides the dilution for the administered (or applied) personal care composition.

The present methods can be used for any suitable purpose. For example, the present methods can be used for cleansing, protecting, smoothing, moisturizing, softening, perfuming, beautifying and/or treating a subject, or a target area, cell, tissue or organ of a subject.

The present methods can be used on any suitable subject. In some embodiments, the subject is animal, e.g., a pet such as a cat or dog. In some embodiments, the subject is a human, e.g., an infant, a child, a teenager, an adult, a senior, a male, a female, a pregnant woman, a healthy human, or a patient.

### D. EXEMPLARY EMBODIMENTS

In exemplary embodiments, the present inventors studied a large number of different combinations of water-soluble polymers and small molecules, thereby designing and making a formulation system to provide for preservative-free personal care composition, such as preservative-free skin care products.

In exemplary embodiments, certain water-soluble substances are used to bind water, to reduce free water in the solution, that is, to control the water in the product by water activity to inhibit microbial growth in desired products. Usually, water activity (AW) is controlled to be less than 0.76, and moisture content is to be maintained less than 52%, generally less than 40%, preferably less than 35%, but usually more than 25%. Under such conditions, microbial growth in many exemplary skin care products are inhibited.

Under the influence of the water-soluble substance on the water binding ability, a substance with strong water binding ability often inhibits the growth of the microorganism even in a composition with higher the moisture content. On the other hand, a substance with weak water binding ability often needs lower water content to inhibit the growth of microorganisms. The size of water activity (AW) often has a direct relationship with the growth of microorganisms. The lower the water activity (AW), the more completely the microbial growth is inhibited. However, the water content and water activity do not correspond completely. Substances with different water solubilities have different water binding capabilities. Under a given moisture content, a solution with a substance having a stronger water binding capability leads to a solution having a smaller AW, which in turn leads to stronger inhibition of microbial growth.

Sometimes, a solution having an AW of 7.5 does not absolutely inhibit the growth of all microorganisms. In some cases, lowering the pH to acidity, increasing pH to alkaline, or using other substances can synergistically inhibit the bacteria and achieve the purpose of preservation.

In exemplary embodiments, water soluble substances that can be used to adjust or control AW include the following categories: water-soluble polymers such as xanthan gum, carboxymethyl cellulose, various modified water-soluble cellulose derivatives, starch, modified starch, carboxymethyl chitin, hydroxypropyl chitosan and various water-soluble shells Glycan, nucleocapsid gum, pullulan, hyaluronic acid, beta-glucan, natto gum, carrageenan, agar, celery, linseed gum, locust bean gum, guar gum, gum arabic, sodium alginate, acacia gum, cassia gum, welan gum and other high-molecular substances dissolved in water.

Exemplary water-soluble small molecules include polysaccharides such as dextrin, malt polysaccharide, glucose, fructose, trehalose, sucrose, honey, erythritol. Exemplary water-soluble small molecules such as glycerin, propylene glycol, sorbitol, maltitol, isomalt, lactitol, mannitol, 1,3-propanediol, 1,3-butanediol, and the like, and various water-soluble polyols, and Water-soluble small molecular substances such as betaine, alginic acid, trehalose, xylitol, and urea can also be used in the present exemplary formulation system. Various other water-soluble substances, but not limited to the above categories, can be used in the above system.

Another feature of the present exemplary embodiment is the use of skin care products. The previous skin care products are often directly applied to the skin. The present exemplary method for using the skin care products can include first spraying the skin with water or first moisturizing the skin with water, and then repeatedly applying the desired amount of the present exemplary formulation system to the skin. In some cases, the exemplary skin care product cannot be directly applied due to high concentration.

Since the exemplary product manufactured by the exemplary method does not use any preservative, and is diluted with water when used, and the skin will continuously release sweat and the like into the skin, the water on the skin is much higher than the moisture of the skin care product itself, thereby significantly increasing the water activity. As such, applying the exemplary skin care product will not significantly affect the growth of microorganisms parasitic on the skin, thus maintaining the microbial ecology of the skin itself. Accordingly, using the present exemplary skin care product helps to restore natural microbiome.

In many cases, using the moisturizer prepared by the present exemplary method effectively treats certain types of skin diseases, without using drugs, substances similar to drugs, or hormones. Not wishing to be bound by any hypothesis or mechanisms discussed herein, the present exemplary moisturizers may help the growth of skin probiotics. The beneficial microbes cooperate with the body's own immune system to fight together, thus helping the skin to heal the skin disease and restore the skin's micro-ecology. In many cases, the treated skin condition does not recur, which is quite different from the hormonal treatment.

Relevant standards have been discussed in connection with the importance of water activity for microbiological monitoring of food and drug. In the section of United States Pharmacopeia (USP)<1112> Application of Water Activity Determination to Nonsterile Pharmaceutical Products, the introduction of the system, the application of water activity in microbial control and testing strategies for non-sterile preparations, and lists the water activities required for common microbial growth, as well as the water activity values of different non-sterile dosage forms and corresponding testing strategies. The USP's new chapter USP<922> Water Activity is also being drafted, with new chapters focusing on water activity testing. USP describes the testing and application of water activity from different perspectives in different chapters.

The water activity required for the growth of some microorganisms is listed in USP<1112>, as shown in Table 1 below:

TABLE 1
Water Activities (a_{w}) Required to Support
the Growth of Representative Microorganisms

| | Water | | Water |
|---|---|---|---|
| | Activity | | Activity |
| Bacteria | (a_{w}) | Molds and Yeast | (a_{w}) |
| Pseudomonas aeruginosa | 0.97 | [symbol] nigricans | 0.93 |
| Bacillus cereus | 0.95 | Mucor plumbeus | 0.92 |
| Clostridium botulinum, Type A | 0.95 | Rhodotorula mucilaginosa | 0.92 |
| Escherichia coli | 0.95 | Saccharomyces cerevisiae | 0.90 |
| Clostridium perfringens | 0.95 | [symbol] | 0.84 |
| [symbol] | 0.95 | Penicillium chrysogenum | 0.83 |
| Salmonella spp. | 0.95 | Aspergillus fumigatus | 0.82 |
| Enterobacter aerogenes | 0.94 | Penicillium glabrum | 0.83 |
| Bacillus subtilis | 0.90 | Aspergillus flavus | 0.78 |
| Micrococcus [symbol] | 0.93 | Aspergillus niger | 0.77 |
| Staphylococcus aureus | 0.86 | Zygosacharomyces rouxii | [symbol] |
| | | (osmophilic yeast) | |
| [symbol] | 0.75 | [symbol] | 0.81 |
| | | (xerophilic fungi) | |

[symbol]

indicates data missing or illegible when filed

From this table, we can see that the water activity required for the growth and reproduction of some Gram-negative bacteria is above 0.91, while the water activity required for the growth and reproduction of some Gram-positive bacteria is above 0.86, and the mold growth and reproduction required water activity is usually above 0.77, and even molds and yeasts that are resistant to water shortage cannot grow when the water activity is 0.60 or less. Therefore, if the water activity of the drug is lower than 0.77, the growth and reproduction of many microorganisms are not supported, and the risk of microbial contamination is decreased.

Described herein is an exemplary formula composition that is safe to skin during use, does not damage the skin flora, and has a preservative effect on the personal care product during storage. The exemplary composition is characterized in that the water activity is controlled in the composition. In exemplary embodiments, different water-soluble small molecules are formulated individually or in combination into different aqueous solutions wherein water activity (AW) is controlled below 0.75.

The AW value of the above exemplary aqueous solutions are determined and the exemplary aqueous solutions are assessed in microbial challenge test. The test results show that it is possible to meet the anti-corrosion requirements of personal care products by controlling its AW value below 0.75.

### E. EXAMPLES

### Example 1. Assessment of Anti-Corrosion Effect of Various Compositions

Components and Aw of combinations Nos. 1-8

Components of combinations Nos. 1-8 and their AW are listed below:
1 #combination: glycerin 22.22%, betaine 22.22%, fructose 22.22%, water 33.33%; hydration degree Aw0.656;
2 #combination: glycerin 22.22%, betaine 22.22%, xylitol 22.22%, water 33.33%; hydration degree Aw0.639;
3 #combination: glycerin 22.22%, fructose 22.22%, xylitol 22.22%, water 33.33%; hydration degree Aw0.712;
4 #combination: fructose 22.22%, betaine 22.22%, xylitol 22.22%, water 33.33%; hydration degree Aw0.676;
5 #combination: fructose 11.11%, betaine 22.22%, xylitol 22.22%, urea 11.11%; water 33.33%; hydration degree Aw0.749;
6 #combination: glycerin 22.22%, fructose 22.22%, trehalose 22.22%, water 33.33%; hydration degree Aw0.760;
7 #combination: glycerin 22.22%, betaine 22.22%, trehalose 11.11%, fructose 11.11%, water 33.33%; hydration degree Aw0.666; and
8 #combination: glycerin 22.22%, sorbitol 22.22%, stevioside 22.22%, water 33.33%; hydration degree Aw0.711.

### Anti-Corrosion Effect Challenge Experiment

### A Medium

Bacterial medium: lecithin Tween 80 nutrient agar (Hai Bo Bio): 24.0 g of medium was added to a 1000 ml Erlenmeyer flask, and heated and boiled and dissolved in 500 ml of distilled water. Autoclave at 121° C. for 20 minutes, set aside.

Fungal medium: potato dextrose medium (PDA), Bengal red agar medium (Beijing Luqiao): Add 18.3 g of medium in a 1000 ml Erlenmeyer flask, dissolve in 500 ml of distilled water, heat to boil until completely dissolved, and extinguish at 121° C. The bacteria were kept for 15 minutes.

Physiological saline: autoclaved at 121° C. for 15 minutes, ready for use.

### B Test Strain

The following strains have been used in the test:
Escherichia Coli, ATCC 8739;
Pseudomonas Aeruginosa, ATCC 9027;
Staphy Lo Coccus Aureus, ATCC 6538;
Candida Albicans, ATCC 10231; and
Aspergiblus Niger, ATCC 16404.

### C Experimental Method

This experiment refers to the United States Pharmacopoeia USP39<51> microbial preservative efficacy test method, that is, weigh 30 g of each test sample in a sterilized sample bottle, adding a quantitative mixed bacterial suspension, the initial bacterial concentration of the bacteria in the sample is 106 cfu/g. The initial concentration of yeast and mold was 105 cfu/g. Then mix well, seal the vial with sealant and place in an incubator at around 28° C. Samples were taken at 7, 14, 21 and 28 days after inoculation.

### D Evaluation Criteria

The logarithmic decrease in the number of bacteria from the initial to the 7th day cannot be less than 1.0; the logarithmic decrease in the number of bacteria from the initial to the 14th day cannot be less than 3.0; and the number of bacteria does not increase from 14 days to 28 days. The total number of molds and yeasts increased from the initial to 7 days, 14 days, and 28 days, and the total number of molds and yeasts did not increase.

### E Analysis of Results

As shown in the Table 2 below, the results of the microbial challenge test conducted on samples of the eight composition preservative systems indicate that the composition passed the challenge test in the exemplary personal care product. Thus, the exemplary personal care composition can exert a good antiseptic effect in the formulation.

TABLE 2
Antiseptic effect of exemplary personal care compositions

| | Different | | | | | | |
|---|---|---|---|---|---|---|---|
| | combination | Total number of colonies/(cfu · g⁻¹) | | | | | |
| Strain | number | Day 0 | Day 1 | Day 7 | Day 14 | Day 21 | Day 28 |
| Mixed | 1# | 1.1 × 10⁶ | 3.6 × 10⁴ | 5.0 × 10³ | 2.5 × 10² | 2.5 × 10² | 1.2 × 10² |
| bacteria | 2# | 1.3 × 10⁶ | 4.2 × 10⁴ | 4.5 × 10³ | 2 × 10² | 2.3 × 10² | 2.3 × 10² |
| | 3# | 6.0 × 10⁶ | 4.2 × 10⁴ | 5.1 × 10³ | 2 × 10² | 3.8 × 10² | 3.85 × 10² |
| | 4# | 3.5 × 10⁶ | 4.2 × 10⁴ | 1.2 × 10⁴ | 4.3 × 10² | 3.0 × 10² | 3.8 × 10² |
| | 5# | 1.0 × 10⁶ | 5.4 × 10⁴ | 1.1 × 10⁴ | 3.0 × 10² | 4.0 × 10² | 4.1 × 10² |
| | 6# | 1.2 × 10⁶ | 4.4 × 10⁴ | 9.0 × 10³ | 4.0 × 10² | 4.0 × 10² | 3.3 × 10² |
| | 7# | 1.0 × 10⁶ | 4.8 × 10⁴ | 8.5 × 10³ | 6.5 × 10² | 3.6 × 10² | 2.85 × 10² |
| | 8# | 1.0 × 10⁶ | 3.5 × 10⁴ | 8.6 × 10³ | 2.5 × 10² | 3.3 × 10² | 3.65 × 10² |
| Mixed | 1# | 1.1 × 10⁶ | 3.5 × 10⁴ | 4.0 × 10³ | 1.3 × 10³ | 2.2 × 10³ | 2.4 × 10³ |
| fungus | 2# | 2.0 × 10⁶ | 3.4 × 10⁴ | 4.0 × 10³ | 1.5 × 10³ | 2.3 × 10³ | 2.1 × 10³ |
| | 3# | 1.3 × 10⁶ | 3.3 × 10⁴ | 5.3 × 10³ | 1.3 × 10³ | 2.1 × 10³ | 2.1 × 10³ |
| | 4# | 3.3 × 10⁶ | 3.0 × 10⁴ | 7.0 × 10³ | 1.8 × 10³ | 1.3 × 10³ | 2.1 × 10³ |
| | 5# | 5.0 × 10⁶ | 3.4 × 10⁴ | 1.2 × 10⁴ | 1.3 × 10³ | 1.6 × 10³ | 2.3 × 10³ |
| | 6# | 4.0 × 10⁶ | 4.2 × 10⁴ | 7.0 × 10³ | 1.1 × 10³ | 9.0 × 10² | 1.6 × 10³ |
| | 7# | 5.0 × 10⁶ | 4.0 × 10⁴ | 7.0 × 10³ | 1.0 × 10³ | 8.0 × 10² | 8.3 × 10² |
| | 8# | 2.0 × 10⁶ | 3.0 × 10⁴ | 7.0 × 10³ | 1.0 × 10³ | 1.2 × 10³ | 2.2 × 10³ |

### Example 2

### Components and Aw of Combinations Nos. 901#-909#

Components of exemplary combinations Nos. 1-9 and their AW are listed below:
901 #combination: glycerin 22.22%, betaine 22.22%, fructose 22.22%, water 33.33%; hydration degree Aw0.666;
902 #combination: fructose 22.22%, betaine 22.22%, xylitol 22.22%, water 33.33%; hydration Aw0.721;
903 #combination: glycerin 22.22%, fructose 22.22%, trehalose 22.22%, water 33.33%; hydration degree Aw0.760;
904 #combination: fructose 22.22%, sorbitol 22.22%, glucose 22.22%, water 33.33%; hydration degree Aw0.734;
905 #combination: fructose 11.11%, betaine 22.22%, glucose 22.22%, water 33.33%; hydration Aw0.0.720;
906 #combination: betaine 22.22%, sorbitol 22.22%, glucose 22.22%, water 33.33%; hydration degree Aw0.719;
907 #combination: glycerin 22.22%, fructose 22.22%, glucose 22.22%, water 33.33%; hydration degree Aw0.742;
908 #combination: betaine 22.22%, fructose 22.22%, trehalose 22.22%, water 33.33%; hydration degree Aw0.749; and
909 #combination: glycerin 22.22%, fructose 22.22%, glucose 22.22%, water 33.33%; hydration degree Aw0.755.

The results of the microbial challenge test conducted on samples of the above 9 exemplary compositions are shown in the Table 3 below.

TABLE 3
Antiseptic effect of exemplary personal care compositions

| | Different | | | | | | |
|---|---|---|---|---|---|---|---|
| | combination | Total number of colonies/(cfu · g⁻¹) | | | | | |
| Strain | number | Day 0 | Day 1 | Day 7 | Day 14 | Day 21 | Day 28 |
| Mixed | 901# | 4.3 × 10⁶ | 3.6 × 10⁴ | <10 | <10 | <10 | <10 |
| bacteria | 902# | 4.0 × 10⁶ | 4.0 × 10⁴ | <10 | <10 | <10 | <10 |
| | 903# | 2.5 × 10⁶ | 1.4 × 10⁶ | 1.2 × 10⁴ | <10 | <10 | <10 |
| | 904# | 4.0 × 10⁶ | 1.0 × 10⁶ | 5.7 × 10⁵ | 1.0 × 10⁴ | <10 | <10 |
| | 905# | 4.0 × 10⁶ | 3.9 × 10⁵ | 3.0 × 10³ | <10 | <10 | <10 |
| | 906# | 3.5 × 10⁶ | 7.3 × 10⁴ | <10 | <10 | <10 | <10 |
| | 907# | 3.8 × 10⁶ | 1.2 × 10⁴ | <10 | <10 | <10 | <10 |
| | 908# | 3.4 × 10⁶ | 3.7 × 10⁴ | <10 | <10 | <10 | <10 |
| | 909# | 3.8 × 10⁶ | 3.5 × 10⁵ | 2.0 × 10³ | <10 | <10 | <10 |
| Mixed | 901# | 3.8 × 10⁵ | 3.3 × 10⁴ | 2.0 × 10³ | 4.7 × 10² | <100 | <10 |
| fungus | 902# | 2.7 × 10⁵ | 4.7 × 10⁴ | <100 | <10 | <10 | <10 |
| | 903# | 1.8 × 10⁵ | 1.7 × 10⁵ | 5.7 × 10⁴ | 1.17 × 10⁴ | 4.8 × 10³ | 1.04 × 10³ |
| | 904# | 3.0 × 10⁵ | 2.5 × 10⁵ | 7.3 × 10⁴ | 5.5 × 10³ | 2.5 × 10³ | 1.3 × 10³ |
| | 905# | 3.8 × 10⁵ | 4.0 × 10⁴ | 1.21 × 10⁴ | 1.4 × 10³ | 1.1 × 10³ | 5.7 × 10³ |
| | 906# | 3.3 × 10⁵ | 1.4 × 10⁵ | 1.3 × 10⁴ | 6.3 × 10³ | 2.9 × 10³ | 1.2 × 10³ |
| | 907# | 4.8 × 10⁵ | 4.1 × 10⁴ | <100 | <10 | <10 | <10 |
| | 908# | 3.5 × 10⁵ | 3.2 × 10⁴ | 8.7 × 10³ | 5.7 × 10³ | 3.3 × 10³ | 1.08 × 10³ |
| | 909# | 4.0 × 10⁵ | 3.8 × 10⁴ | 1.4 × 10⁴ | 5.7 × 10³ | 7.0 × 10³ | 1.1 × 10³ |

### Example 3

Components of exemplary combinations Nos. 1-7 and their AW are listed Table 4 below.

TABLE 4
Components of exemplary combinations Nos. 1-7 and their AW

| Different | | | | Water |
|---|---|---|---|---|
| combination | Glycerin | Betain | water | Activity |
| number | (m %) | (m %) | (m %) | Aw |
| 1 | 71 | 0 | 29 | 0.638 |
| 2 | 60 | 0 | 40 | 0.735 |
| 3 | 0 | 60 | 40 | 0.569 |
| 4 | 0 | 54 | 46 | 0.662 |
| 5 | 0 | 48 | 52 | 0.742 |
| 6 | 15 | 40 | 45 | 0.712 |
| 7 | 27.5 | 27.5 | 45 | 0.744 |

The results of the microbial challenge test conducted on samples of the above 7 exemplary compositions are shown in the Table 5 below.

TABLE 5
Antiseptic effect of exemplary personal care compositions

| | Different | | | | | | |
|---|---|---|---|---|---|---|---|
| | combination | Total number of colonies/(cfu · g⁻¹) | | | | | |
| Strain | number | Day 0 | Day 1 | Day 7 | Day 14 | Day 21 | Day 28 |
| Mixed | 1 | 3.7 × 10⁶ | 8.6 × 10⁴ | <100 | <10 | <10 | <10 |
| bacteria | 2 | 6.4 × 10⁶ | 5.8 × 10⁴ | 8.5 × 103 | 5.8 × 10² | 1.1 × 10² | <100 |
| | 3 | 4.1 × 10⁶ | 7.4 × 10⁴ | <100 | <10 | <10 | <10 |
| | 4 | 5.1 × 10⁶ | 6.2 × 10⁴ | <100 | <10 | <10 | <10 |
| | 5 | 4.7 × 10⁶ | 5.4 × 10⁴ | 5.6 × 103 | 3.0 × 10² | <100 | <10 |
| | 6 | 2.6 × 10⁶ | 8.9 × 10⁴ | <100 | <10 | <10 | <10 |
| | 7 | 2.3 × 10⁶ | 8.9 × 10⁴ | 5.2 × 10³ | 2.3 × 10² | <100 | <10 |
| Mixed | 1 | 3.2 × 10⁵ | 3.3 × 10⁴ | 2.3 × 10² | <100 | <10 | <10 |
| fungus | 2 | 2.5 × 10⁵ | 3.4 × 10⁴ | 1.3 × 10³ | 6.5 × 10² | 2.3 × 10² | <100 |
| | 3 | 3.4 × 10⁵ | 3.5 × 10³ | <100 | <10 | <10 | <10 |
| | 4 | 4.5 × 10⁵ | 6.8 × 10⁴ | <100 | <10 | <10 | <10 |
| | 5 | 4.5 × 10⁵ | 6.8 × 10⁴ | 1.2 × 10³ | 7.8 × 10² | 1.1 × 10² | <100 |
| | 6 | 5.0 × 10⁵ | 5.6 × 10⁴ | 2.1 × 10³ | 1.1 × 10³ | 5.2 × 10² | 1.1 × 10² |
| | 7 | 5.8 × 10⁵ | 4.5 × 10⁴ | 7.0 × 10³ | 1.5 × 10³ | 8.0 × 10² | 3.8 × 10² |

It can be seen from the above that the solution below Aw0.75 can meet the microbial corrosion protection requirements.

It should be noted that products in the range of Aw0.65-0.75 may also have microbial growth in some extreme cases, but through the product transfer or using weak acidity (such as PH value less than 5) or weakly alkaline (such as The PH value is greater than 8), or other substances with certain antibacterial effects (but not as strong as non-preservatives) can synergistically achieve the purpose of antiseptic.

### Example 4. Determination of Water Activity of Small Molecule Water-Soluble Substances and Added Polymer Compounding

As an example, in a small molecule aqueous solution satisfying Aw0.75, different proportions of the polymer are added, and the Aw hydration degree is expected to be equal to or less than the Aw value of the small molecule aqueous solution, thereby also satisfying the antiseptic requirements of the skin care product.

### Example 5. No Anti-Corrosion System Design of Cream Emulsion

An aqueous solution satisfying Aw less than 0.75 can be used as the aqueous phase. After compounding with different proportions of grease and emulsifier, it can still be expected to meet the requirement of Aw less than 0.75. The Aw value will only be lower than the Aw of the aqueous phase after compounding, thus making it more capable of meet the anti-corrosion requirements of the product.

### Example 6. Examples of Preparation of Personal Care Skin Care Products without Preservatives

### An Exemplary Preservative-Free Skin Care Essence

An exemplary preservative-free skin care essence contains hyaluronic acid in a range of 0.1-1.0% (w/w), glycerol in a range of 55-70% (w/w), lactic acid bacteria fermentation broth in a range of 1-39% (w/w), and water in a range of 25-44% (w/w). The exemplary preservative-free skin care essence has Aw in a range of 0.60-0.75. The exemplary system can meet the microbial challenge test to achieve the anti-corrosion requirements for cosmetics.

One specific exemplary preservative-free skin care essence contains hyaluronic acid 0.4% (w/w), glycerol 68% (w/w), lactic acid bacteria fermentation broth 20% (w/w) wherein at least 90% is water, and water 10% (w/w). The specific exemplary preservative-free skin care essence has Aw 0.665. The specific exemplary preservative-free skin care essence can meet the microbial challenge test, can meet the anti-corrosion requirements.

### An Exemplary Preservative Skin Care Essence: Aloe Vera Extract

An exemplary preservative-free skin care essence contains glycerin in a range of 10-30% (w/w), betaine in a range of 10-40% (w/w), trehalose in a range of 10-30% (w/w), aloe juice in a range of 1-45% (w/w) wherein at least 90% is water, and fermentation broth in a range of 1-25% (w/w) wherein at least 90% is water. The exemplary preservative-free skin care essence has Aw in a range of 0.6-0.75, and can meet the requirements of no anti-corrosion addition.

An Exemplary Natural Skin Care Lotion without Preservative Added: Shea Butter Moisturizing Skin Lotion

Aqueous phase of an exemplary natural skin care lotion contains glycerin in a range of 5-30% (w/w), betaine in a range of 5-30% (w/w), xylitol in a range of 5-20% (w/w), fructose in a range of 5-30% (w/w), a plant extract aqueous solution in a range of 30-40% (w/w) wherein at least 90% is water, and xanthan gum in a range of 0.1-0.5% (w/w).

Oil phase of an exemplary natural skin care lotion contains soy lecithin in a range of 1-2% (w/w), shea butter in a range of 5-15% (w/w), olive oil in a range of 5-15% (w/w), and sea buckthorn oil in a range of 1-10% (w/w).

The water phase and the oil phase are heated to 75° C. respectively, then the water phase is stirred in the oil phase for 10 minutes, then homogenized for three minutes, and then stirred and cooled to 40° C., then placed in a bucket and allowed to stand still for one night to eliminate bubbles, then canned packaging into products. This exemplary system has Aw less than 0.75.

An Exemplary Natural Skin Care Lotion without Preservative Added: Olive Oil Moisturizer

Aqueous phase of an exemplary natural skin care lotion contains glycerin in a range of 10-50% (w/w), xylitol in a range of 5-20% (w/w), trehalose in a range of 5-30% (w/w), a plant fermentation aqueous solution in a range of 30-40% (w/w) wherein at least 90% is water, and hyaluronic acid in a range of 0.1-0.5% (w/w).

Oil phase of an exemplary natural skin care lotion contains monoacylglyceride in a range of 1-2% (w/w), 16-18 carbon mixed fatty alcohol in a range of 5-10% (w/w), olive oil in a range of 5-15% (w/w), avocado oil in a range of 1-10% (w/w).

The water phase and the oil phase are heated to 75° C. respectively, then the water phase is stirred in the oil phase for 10 minutes, then homogenized for three minutes, and then stirred and cooled to 40° C., then placed in a bucket and allowed to stand still for one night to eliminate bubbles, then canned packaging into products.

### An Exemplary Preservative-Free Starch Facial Cleanser

An exemplary preservative-free starch facial cleanser contains glycerin in a range of 10-30 g, betaine in a range of 50-100 g, xylitol in a range of 5-50 g, modified starch in a range of 5-20 g, starch in a range of 5-15 g, xanthan gum in a range of 0.1-0.5 g, water in a range of 65-85 g. This exemplary preservative-free starch facial cleanser contains water in a range of 25%-52% (w/w) and has Aw less than 0.75.

Stir at 75° C. for 40 minutes, gradually reduce the temperature to 35° C., store in a bucket for 24 hours, and then fill the finished product. This exemplary preservative-free starch facial cleanser has Aw less than 0.75.

### Example 7. An Exemplary No-Surfactant Toothpaste

An exemplary no-surfactant toothpaste contains Xanthan gum 0.3 g, starch 6.0 g, calcium carbonate 15 g, plant extract 3 g, glycerin 25 g, betaine 30 g, and water 30 g. The above ingredients are evenly mixed, stirred at 75° C., homogenized for three minutes, then lowered to 40° C., and the cans are finished. This exemplary no-surfactant toothpaste has Aw 0.619.

1. A personal care composition, which composition comprises:
1) a personal care substance; and
2) a water activity adjusting substance,
wherein said personal care composition comprises from about 25% (w/w) water to about 60% (w/w) water, and said personal care composition has a water activity of 0.76 or less.

2. The personal care composition of claim 1, wherein the water activity adjusting substance is:
1) a small molecule, e.g., a water-soluble small molecule or a water-insoluble small molecule;
2) a polymer, e.g., a water-soluble polymer or a water-insoluble polymer; and/or
3) a combination of 1) and 2).

3-10. (canceled)

11. The personal care composition of claim 1, wherein the water activity adjusting substance is glucose, fructose, trehalose, sucrose, honey, erythritol, glycerin, propylene glycol, sorbitol, maltitol, isomalt, lactitol, mannitol, 1,3-propanediol, 1,3-butanediol, betaine, alginic acid, trehalose, xylitol, urea, a water-soluble polyol, or a polysaccharide, e.g., dextrin or malt polysaccharide.

12. The personal care composition of claim 1, wherein the water activity adjusting substance has a level or concentration from about 0.01% (w/w) to about 75% (w/w).

13. The personal care composition of claim 1, which comprises from about 25% (w/w) water to about 52% (w/w) water.

14. The personal care composition of claim 1, which has a water activity from about 0.5 to about 0.76.

15. The personal care composition of claim 1, which comprises a preservative content, e.g., an antimicrobial preservative content, that is from about one tenth to about one fifth of the preservative content that is needed in a corresponding personal care composition that has a water activity of 0.8 or more.

16. The personal care composition of claim 1, which does not comprise a preservative e.g., an antimicrobial preservative, or does not comprise a chemically synthesized preservative e.g., a chemically synthesized antimicrobial preservative.

17. The personal care composition of claim 1, which has a surfactant content of 8% (w/w) or less.

18. The personal care composition of claim 1, which does not comprise a surfactant, e.g., a chemically synthesized surfactant.

19. The personal care composition of claim 1, which further comprises a stabilizing substance that reduces or prevents precipitation, sedimentation or crystallization of a component of the personal care composition.

20. The personal care composition of claim 1, wherein the personal care substance comprises a natural substance or a substance that is not chemically synthesized.

21. The personal care composition of claim 1, wherein the personal care substance has a level or concentration from about 0.01% (w/w) to about 75% (w/w).

22. The personal care composition of claim 1, which is configured to have cleansing, protecting, e.g., protecting against sun burn, smoothing, moisturizing, softening, perfuming, beautifying and/or treating function.

23. The personal care composition of claim 1, which is configured to be a skin care, hair care, oral care, shaving and/or grooming, sun care, deodorant and/or antiperspiran, bath and/or body, incontinence, pregnancy and/or fertility, feminine care, eye care, ear, nose and/or throat care, foot care, lip care, or massage and/or relaxation composition or product.

24. The personal care composition of claim 1, which is configured to be a cosmetic or make-up composition or product.

25. The personal care composition of claim 1, which does not comprise a preservative content, e.g., an antimicrobial preservative, and does not comprise a surfactant.

26. The personal care composition of claim 1, which is self-preserving.

27. The personal care composition of claim 1, which does dot comprise any substance that is chemically synthesized.

28. The personal care composition of claim 1, which is configured, when applied to a subject, to have reduced or no damage to the subject and/or the subject's cell, tissue organ.

29. The personal care composition of claim 1, which is configured, when applied to a subject, to have reduced or no damage to the subject's microbiome (or microbiota).

30. The personal care composition of claim 1, which is in the form of a liquid, a spray, a cream, an ointment, a liniment (balm), a paste or a film.

31. The personal care composition of claim 1, which is configured to be a concentrate that needs to be diluted for use, e.g., needs to be diluted using water or an aqueous liquid for use.

32. A kit, a package or an article of manufacture, which comprises a personal care composition of claim 1.

33. A method, which comprises administering (or applying) a personal care composition of claim 1 to a subject.
